(19) United States
(12) Patent Application Publication
(10) Pub. No.: US 2022/0303634 A1
(43) Pub. Date: Sep. 22, 2022

(54) SYSTEM AND METHOD FOR PROVIDING HEALTH ALERTS IN RESPONSE TO VIDEO CONTENT

(71) Applicant: ARRIS ENTERPRISES LLC, Suwanee, GA (US)

(72) Inventors: Jianguo ZHOU, Shanghai (CN); Fenggen HU, Shanghai (CN); Yuyang ZHANG, Shanghai (CN)

(73) Assignee: ARRIS ENTERPRISES LLC, Suwanee, GA (US)

(21) Appl. No.: 17/428,542

(22) PCT Filed: Aug. 24, 2020
(86) PCT No.: PCT/CN2020/110776

§ 371 Date: Aug. 4, 2021

(51) Int. Cl. H04N 21/488; H04N 21/81; H04N 21/422

(52) U.S. Cl. CPC H04N 21/4882; H04N 21/812; H04N 21/42201

(57) ABSTRACT

A system and method for alerting an individual viewing a video advertisement that the advertised product may be incompatible with the viewer's health profile. This incompatibility may be a consequence of a specific allergy, sensitivity or other interaction or reaction that the advertised product may cause. The system and method utilize previously stored viewer health profiles. These profiles may be associated with a wearable health monitor, or can be entered by a viewer specifically to support the operation of the disclosed system. Viewers may be identified through the detection of a wearable health monitor, or via facial recognition.

### BACKGROUND OF THE INVENTION

In today's media-rich social environment, viewers are typically exposed to media from multiple sources via many screens (television, tablets, computers, smartphones, etc.). The content viewed on these screens is often accompanied by numerous commercial advertisements ranging from traditional television commercials to product placement and pop-up ads.

The subject matter of these advertisements can relate to every type of product or service imaginable, but a significant number of them are focused on items that a individual would ingest or apply to their person. These items would include, but are limited to, food, beverages, over-the-counter and prescription medicines, soaps, shampoos, creams and lotions. The advertisements for these ingested and applied products (“IA products”) may include warning notices alerting an individual to possible detrimental side effects, reactions or interactions, but such warnings are typically included due to a government mandate and appear most often in prescription drug advertisements. The majority of IA product ads are presented to individuals without any warnings, and certainly none tailored to a particular individual's state of health, allergic sensitivities or risk of possible drug interactions. If an individual is allergic to nuts, an advertisement for nuts will not typically provide a warning to that individual advising them that consuming the advertised product could be detrimental to their health.

More than ever before, individuals are taking steps to monitor their own health and well-being by utilizing wearable health monitor (“WHM”) devices. These devices include smartwatches and wearable fitness monitors. Such devices can monitor and record the wearer's activities, heart rate, temperature, blood oxygen level, etc. In addition to monitoring the health and activity of a wearer, these WHM devices also provide important medical information that may be needed if the wearer is in need of urgent care. This information would typically include the user's height, weight, age, food and drug allergies, prescription drug history, blood type, emergency contact information, etc. A user would typically enter this medical information, utilizing an interface such as a computer or smartphone. This information would then be compiled to create a personal health profile that would be downloaded to the WHM device, or uploaded to a remote server that could be accessed utilizing the WHM device. This information in the health profile can then be utilized to enable the WHM device to provide the wearer with alerts and guidance based upon their particular health and medical information, and/or accessed to provide medical professionals with critical information if the wearer is in an emergency situation.

Given the widespread use of WHM devices, it would be desirable for a media system to access the associated health and medical information for given individual and proactively generate an on-screen warning during an IA product advertisement. The warning would ideally notify the individual that the consumption, application or use of the advertised IA product could be detrimental to their health and well-being.

### BRIEF SUMMARY OF THE INVENTION

A system and method for alerting an individual viewing a video advertisement that the advertised product may be incompatible with the viewer's health profile. This incompatibility may be a consequence of a specific allergy, sensitivity or other interaction or reaction that the advertised product may cause. The system and method utilize previously stored viewer health profiles. These health profiles may be associated with a wearable health monitor, or can be entered by a viewer specifically to support the operation of the disclosed system. Viewers may be identified through the detection of a wearable health monitor, or via facial recognition.

### BRIEF DESCRIPTION OF THE DRAWINGS

The aspects, and advantages of the present invention will become better understood with regard to the following description, appended claims, and accompanying drawings in which:

FIG. 1 is a is a functional block diagram of a preferred embodiment of a system adapted to generate and display user-specific health warnings in response to and concurrent with product advertisements.

FIG. 2 is a flow diagram of a process for generating and displaying user-specific health warnings in response to and concurrent with product advertisements utilizing the system of FIG. 1.

FIG. 3 is a depiction of a first video advertisement and a single, user-specific health warning displayed concurrently therewith.

FIG. 4 is a depiction of a second video advertisement and two separate user-specific health warnings displayed concurrently therewith.

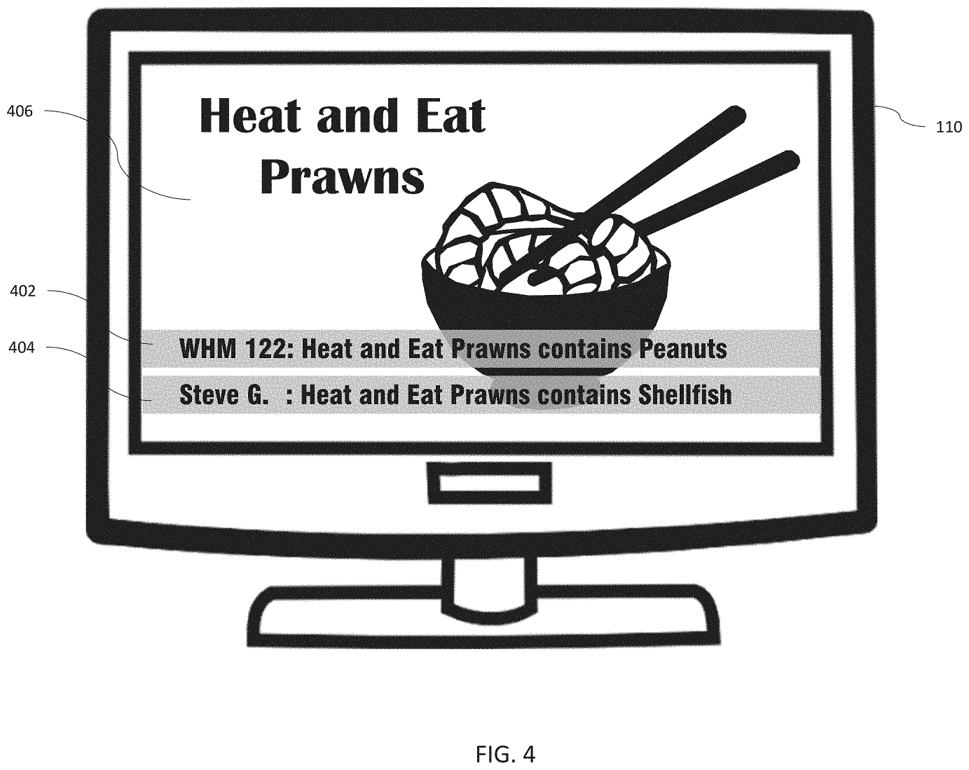

FIG. 5 is a is a functional block diagram of an alternate preferred embodiment of a system adapted to generate and display user-specific health warnings in response to and concurrent with product advertisements.

FIG. 6 is a flow diagram of a process for generating and displaying user-specific health warnings in response to and concurrent with product advertisements utilizing the system of FIG. 5.

### DETAILED DESCRIPTION

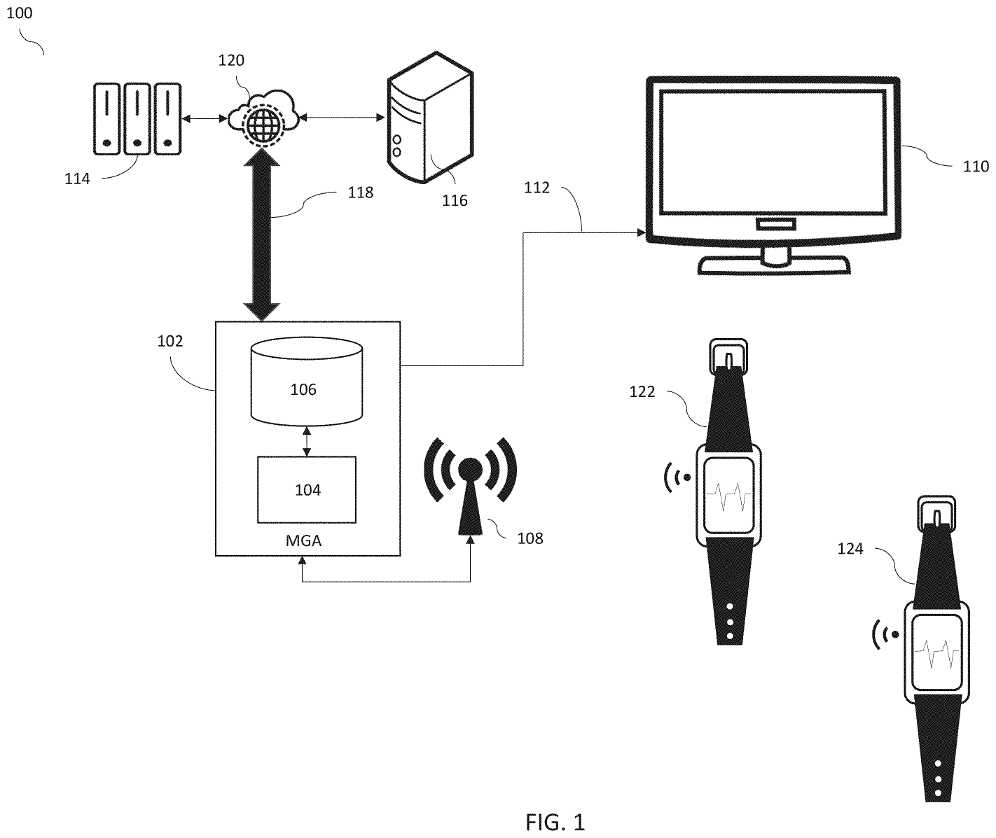

FIG. 1 is a functional diagram of a preferred embodiment of a system (100) adapted to concurrently display viewer-specific health-related warnings in response to video advertisements of IA products. As shown, system 100 comprises media gateway appliance (“MGA”) 102, which includes processor 104, memory 106 and wireless transceiver 108. MGA 102 is also shown to be linked to digital television 110 (via HDMI cable 112) and linked to headend 114 and remote server 116 (via broadband link 118 and network 120). WHM devices 122 and 124 are also shown. Each of these devices is linked to MGA 102 via a wireless network supported by wireless transceiver 108.

MGA 102 functions as a primary appliance for the transmission and reception of digital data in a residential environment. This data can include e-mail, video (streaming and recorded), audio, images and network television. One example of an MGA is a set-top box, typically provided to an end user by a cable, optical, or satellite multiservice operator (“MSO”). A user may utilize television 110 to view video or images received by or stored within MGA 102.

WHM devices 122 and 124 can be dedicated wearable appliances functioning exclusively to monitor a wearer's health and activity, or they could smartwatches providing a wearer with health and activity monitoring functionality. In addition to monitoring the health and activity of a wearer, the WHM devices can also serve to provide medical information to medical providers if the wearer is in need of urgent care. This information would typically include the user's food and drug allergies, blood type, emergency contact information, etc. This critical medical information is typically stored in a user-compiled health profile stored within or accessible via each of WHM devices (122, 124).

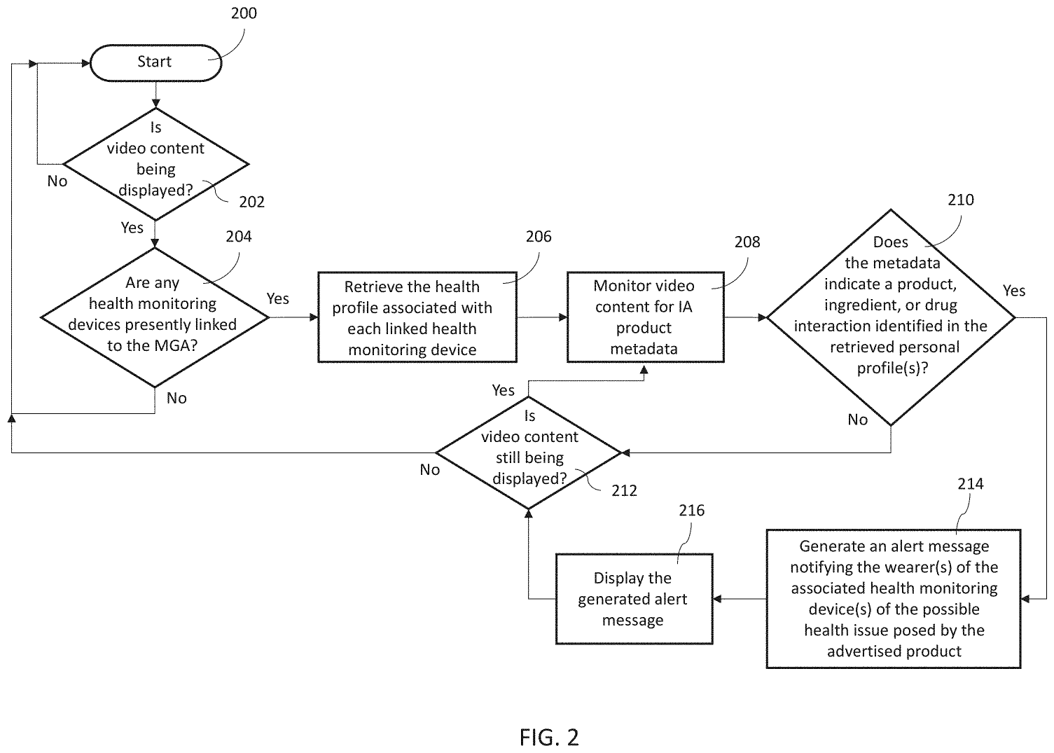

In a particular embodiment, processor 104 determines if video content is being displayed upon television 110 (steps 200 and 202). If not, the process loops back to step 200. If, however video is being displayed, the process continues with processor 104 determining if one or more WHM devices are presently linked to MGA 102 via wireless transceiver 108 (see FIG. 2, step 204). If no such WHM devices are detected, the process loops back to step 204 and processor 104 continues to search for such. If, as shown in FIG. 1, processor detects that WHM devices 122 and 124 are linked to MGA 102, processor 104 accesses and retrieves the health profile associated with each WHM device (step 206). This retrieval could be made directly from information stored in each of the WHM devices (122, 124), or from information stored in a remote server (116). The particular information identifying the remote server having been retrieved from a WHM device. The retrieved information can then be stored locally in memory 106 to facilitate access by processor 104. As shown in FIG. 1, WHM devices 122 and 124 are both linked to MGA 102 via wireless transceiver 108. Consequently, processor 104 would retrieve the health profile associated with each of the devices.

Processor 104 would then monitor any video being displayed by MGA 102 on television 110 for the inclusion of IA product metadata (step 208). If no such metadata is detected (a negative outcome of step 210), the process continues with step 212 and processor 104 determines if video content is still being displayed on television 110. If so the process loops to step 208 and the processor continues to monitor for IA product metadata. If the video is found to have terminated, the process continues with step 200 and processor 104 senses if new video content is being displayed (step 202). However, if in step 210 it was determined that the metadata indicated a particular IA product, ingredient or interaction corresponding to either of the retrieved health profiles, the process continues with step 214. In step 214, utilizing information stored in memory 106, and/or retrieved from headend 114, processor 104 generates one or more messages for display upon television 110. These messages would be displayed concurrently with the advertisement associated with the metadata (step 216). The messages could be displayed as a pop-up, a crawler, or as a picture-in-picture superimposed on the advertisement. The message content would specify the possible health issue related to the advertised product. The message would also identify the particular WHM device (or, based upon information stored in memory 106, the identity of the individual wearing the device) to which the message pertained.

With respect to system 100 in FIG. 1, assume that, as shown in Table A, processor 104 retrieved the following health profile information for WHM 122 and 124:

TABLE A

| WHM 122 | WHM 124 |
|---|---|
| Health Profile | Health Profile |
| Allergic to: | Allergic to: |
| Peanuts | Shellfish |
| Tree Pollen | |
| Prescriptions: | Prescriptions: |
| None | Simvastatin |
| | 40 mg |

Additionally, assume that a particular IA product advertisement was associated with the metadata shown in Table B:

TABLE B
Product Name: Wheat Flakes

Ingredients:
Wheat Flour
Sugar
Peanut Oil

Given the above information, processor 104 would recognize the ingredient “Peanut Oil” as related to the allergen “Peanuts”. The process enabling this recognition could be performed by processor 104, or performed at the request of processor 104 by a remote system accessible via network 120. Utilization of remote resources could allow for expert and or AI systems to be leveraged, and permit access to greater computing resources and data bases so as to increase the speed and accuracy possible detrimental products, ingredients or interactions. Once the ingredient “Peanut Oil” was identified as a potential allergen for the wearer of WHM device 122, processor 104 would generate and cause to be displayed upon television 110 display a message conveying the following information:

WHM 122: Product Wheat Flakes contains Peanut Oil

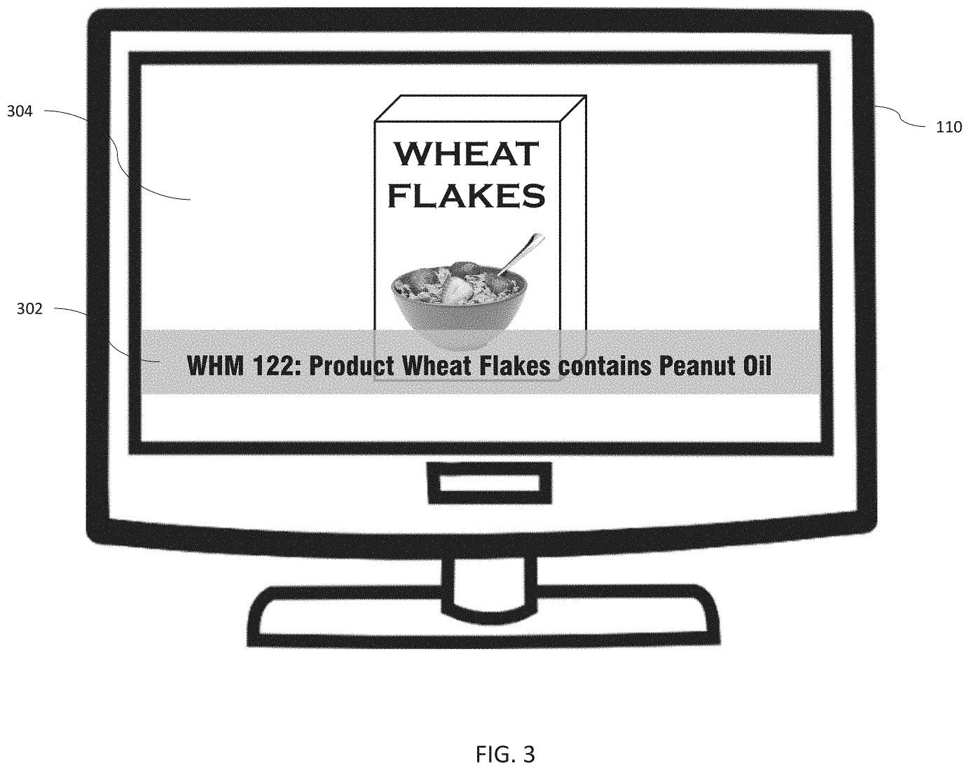

FIG. 3 provides an illustration of how the message could be displayed concurrently with the advertisement for the Wheat Flakes product. As shown the message 302 is superimposed upon the advertisement video (304) being viewed on television 110. This message could be displayed for a predetermined period, or terminate when the related advertisement terminated.

If the product being advertised had ingredients which were identified as relating to the health profiles of both WHM 122 and 124, processor 104 would generate two messages. Assume that a particular commercial was advertising a prepared dish called “Heat and Eat Prawns that included crushed peanuts and prawns. The crushed peanuts would be identified as possibly detrimental to the person associated with the WHM 122 health profile (peanut allergy), and the prawns as possibly detrimental to the person associated with the WHM 124 health profile (shellfish allergy). Two messages (402 and 404) would be displayed as superimposed upon the product advertisement (406). One would be directed to WHM 122 referencing “peanuts” and other to WHM 124 referencing “prawns”. In the case of the WHM 124 message, processor 104 retrieved the name of the user (“Steve G”) from the health profile and consequently displayed it in the on-screen message rather than “WHM 124”.

In addition, processor 104 could be further adapted to generate audio messages or signals in conjunction with or in lieu of the displayed messages described above. For example, as a message is displayed a brief alert tone could be generated and played through an audio transducer associated with television 110. Such a tone to serve to focus a viewer's attention to the screen so that they would not miss the displayed message. Processor 104 could also be programmed to generate synthesized speech announcing the message as it is displayed, or in lieu of it being displayed.

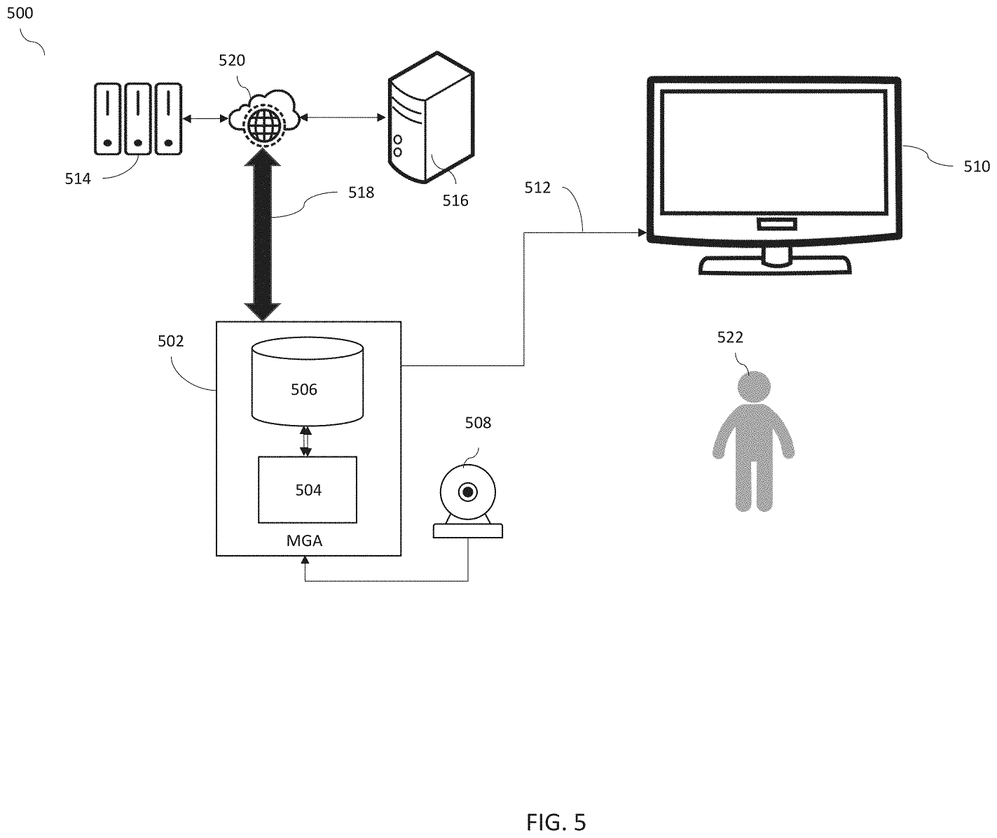

FIG. 5 is a functional diagram of a preferred embodiment of a system (100) adapted to concurrently display viewer-specific health-related warnings in response to video advertisements of IA products. As shown, system 500 comprises media gateway appliance (“MGA”) 502, which includes processor 504, memory 506 and camera 508. MGA 502 is also shown to be linked to digital television 510 (via HDMI cable 512) and linked to headend 514 and remote server 516 (via broadband link 518 and network 520). WHM devices 522 and 524 are also shown. Each of these devices is linked to MGA 502 via a wireless network supported by wireless transceiver 508.

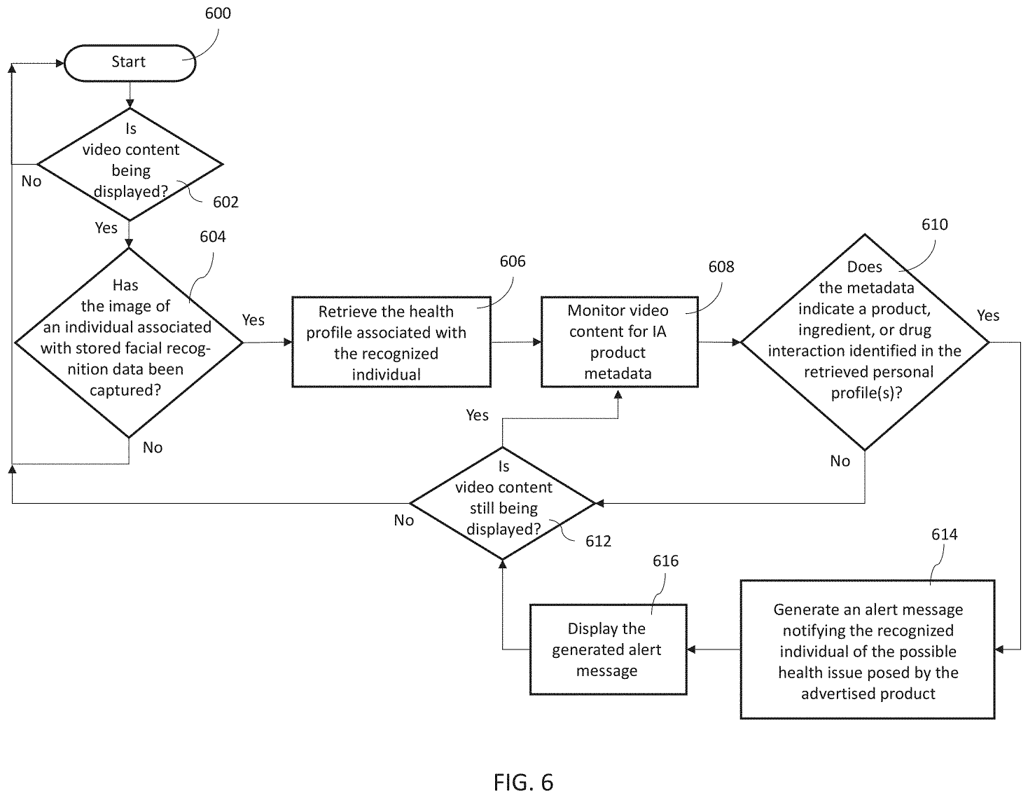

Memory 506 stores critical medical information for one or more individuals (including individual 522). Memory 506 also stores facial recognition data associated with one or more individuals (including individual 522). The system operates in a manner similar to that od system 100 shown in FIG. 1 (see steps 600 and 602 of FIG. 6). However, processor 504 utilizes images (still or video) captured by camera 508 as a means of identifying one or more viewers of television 510 based upon the facial recognition data stored in memory 506 (step 604). If the image data of an individual is determined to be a match for the facial recognition data stored in memory 506, processor 504 retrieves the health profile associated with that individual each WHM device (step 606). This retrieval could be made directly from information stored in memory 506, or from information stored in a remote server (516).

Processor 504 would then monitor any video being displayed by MGA 502 on television 510 for the inclusion of IA product metadata (step 508). If no such metadata is detected (a negative outcome of step 610), the process continues with step 612 and processor 504 determines if video content is still being displayed on television 510. If so the process loops to step 608 and the processor continues to monitor for IA product metadata. If the video is found to have terminated, the process continues with step 600 and processor 504 senses if new video content is being displayed (step 602). However, if in step 610 it was determined that the metadata indicated a particular IA product, ingredient or interaction corresponding to either of the retrieved health profiles, the process continues with step 614. In step 614, utilizing information stored in memory 506, and/or retrieved from headend 514, processor 504 generates one or more messages for display upon television 510. These messages would be displayed concurrently with the advertisement associated with the metadata (step 616). The messages could be displayed as a pop-up, a crawler, or as a picture-in-picture superimposed on the advertisement. The message content would specify the possible health issue related to the advertised product. The message would also identify the particular WHM device (or, based upon information stored in memory 506, the identity of the individual wearing the device) to which the message pertained.

Although the invention herein has been described with reference to particular embodiments, it is to be understood that these embodiments are merely illustrative of the principles and applications of the present invention. For example, the MGA can be a stand-alone device, or integrated into another system or device such as a television, a digital assistant, smartphone, tablet or a computer. In addition, the alert messages and/or tones generated by the system could be provided via an individual's WHM instead of or in conjunction with the provision of such on a television screen. WHM alert messages can also include the generation of a vibration by the WHM.

1. A system for generating a concurrent alert in response to a displayed video advertisement, the system comprising:
at least one media gateway appliance, comprising at least one processor, at least one memory, and at least one wireless signal receiver, wherein the memory is adapted to store data identifying at least one wearable health monitor associated with at least one health profile; and
wherein the at least one processor is adapted to:
identify at least one wearable health monitor based upon signals received via the at least one wireless signal receiver and the data stored in the at least one memory;
retrieve at least one health profile based upon the identification of the at least one wearable health monitor;
detect metadata associated with a displayed video advertisement;
generate an alert message based, at least in part, on the detected metadata and the retrieved at least one heath profile; and
display the alert message concurrently with the video advertisement.

2. The system of claim 1 wherein the at least one health profile is retrieved from at least one of the following:
the at least one wearable health monitor;
the at least one memory; and
a server linked to the at least one media gateway device.

3. The system of claim 1 wherein the alert message comprises a video message identifying at least one of the following:
the at least one wearable health monitor;
the identity of an individual associated with the at least one wearable health monitor;
at least one product associated with the alert message;
at least one ingredient associated with the alert message;
at least one reaction associated with the alert message;
at least one allergy associated with the alert message;
at one interaction associated with the alert message; and
at least one drug.

4. The system of claim 1 wherein the alert message is displayed upon at least one of the following:
a television;
a smartphone;
a computer display;
a tablet; and
the at least one wearable health monitor.

5. The system of claim 1 wherein the at least one processor and the memory are located remotely from the at least one media gateway appliance.

6. A method for generating a concurrent alert in response to a displayed video advertisement, the system comprising:
at least one camera adapted to capture images of individuals viewing the displayed video advertisement; and
at least one media gateway appliance, comprising at least one processor, at least one memory, and at least one wireless signal receiver, wherein the memory is adapted to store facial recognition data associated with at least one individual and at least one health profile; and
wherein the at least one processor is adapted to:
identify at least one individual viewing the displayed video advertisement based, at least in part, on at least one image captured by the at least one camera and the facial recognition data stored in the at least one memory;
retrieve at least one health profile based upon the identification of the at least one individual;
detect metadata associated with a displayed video advertisement;
generate an alert message based, at least in part, on the detected metadata and the retrieved at least one heath profile; and
display the alert message concurrently with the video advertisement.

7. The system of claim 6 wherein the at least one health profile is retrieved from at least one of the following:
the at least one memory; and
a server linked to the at least one media gateway device.

8. The system of claim 6 wherein the alert message comprises a video message identifying at least one of the following:
the at least one identified individual;
at least one product associated with the alert message;
at least one ingredient associated with the alert message;
at least one reaction associated with the alert message;
at least one allergy associated with the alert message;
at one interaction associated with the alert message; and
at least one drug.

9. The system of claim 6 wherein the alert message is displayed upon at least one of the following:
a television;
a smartphone;
a computer display;
a tablet; and
at least one wearable device.

10. The system of claim 6 wherein the at least one processor and the memory are located remotely from the at least one media gateway appliance.

11. A method for generating a concurrent alert in response to a displayed video advertisement, in a system comprising:
at least one media gateway appliance, comprising at least one processor, at least one memory, and at least one wireless signal receiver, wherein the memory is adapted to store data identifying at least one wearable health monitor associated with at least one health profile;
comprising the steps of:
identifying at least one wearable health monitor based upon signals received via the at least one wireless signal receiver and the data stored in the at least one memory;
retrieving at least one health profile based upon the identification of the at least one wearable health monitor;
detecting metadata associated with a displayed video advertisement;
generating an alert message based, at least in part, on the detected metadata and the retrieved at least one heath profile; and
displaying the alert message concurrently with the video advertisement.

12. The method of claim 11 wherein the at least one health profile is retrieved from at least one of the following:
the at least one wearable health monitor;
the at least one memory; and
a server linked to the at least one media gateway device.

13. The method of claim 11 wherein the alert message comprises a video message identifying at least one of the following:
the at least one wearable health monitor;
the identity of an individual associated with the at least one wearable health monitor;
at least one product associated with the alert message;
at least one ingredient associated with the alert message;
at least one reaction associated with the alert message;
at least one allergy associated with the alert message;
at one interaction associated with the alert message; and
at least one drug.

14. The method of claim 11 wherein the alert message is displayed upon at least one of the following:
a television;
a smartphone;
a computer display;
a tablet; and
the at least one wearable health monitor.

15. The method of claim 11 wherein the at least one processor and the memory are located remotely from the at least one media gateway appliance.

16. A method for generating a concurrent alert in response to a displayed video advertisement, in a system comprising:
at least one camera adapted to capture images of individuals viewing the displayed video advertisement; and
at least one media gateway appliance, comprising at least one processor, at least one memory, and at least one wireless signal receiver, wherein the memory is adapted to store facial recognition data associated with at least one individual and at least one health profile;
comprising the steps of:
identifying at least one individual viewing the displayed video advertisement based, at least in part, on at least one image captured by the at least one camera and the facial recognition data stored in the at least one memory;
retrieving at least one health profile based upon the identification of the at least one individual;
detecting metadata associated with a displayed video advertisement;
generating an alert message based, at least in part, on the detected metadata and the retrieved at least one heath profile; and
displaying the alert message concurrently with the video advertisement.

17. The method of claim 16 wherein the at least one health profile is retrieved from at least one of the following:
the at least one memory; and
a server linked to the at least one media gateway device.

18. The method of claim 16 wherein the alert message comprises a video message identifying at least one of the following:
the at least one identified individual;
at least one product associated with the alert message;
at least one ingredient associated with the alert message;
at least one reaction associated with the alert message;
at least one allergy associated with the alert message;
at one interaction associated with the alert message; and
at least one drug.

19. The method of claim 16 wherein the alert message is displayed upon at least one of the following:
a television;
a smartphone;
a computer display;
a tablet; and
at least one wearable device.

20. The method of claim 16 wherein the at least one processor and the memory are located remotely from the at least one media gateway appliance.
